(19) United States
(12) Patent Application Publication
(10) Pub. No.: US 2012/0276217 A1
(43) Pub. Date: Nov. 1, 2012

(54) DISINFECTING FORMULATION

(75) Inventors: Peter Lawrence Steve, Neilborough (AU); Steven Craig Frank, Aberfeldie (AU)

(73) Assignee: SUNNYWIPES PTY LTD, NORTHMEAD (AU)

(21) Appl. No.: 12/989,251

(22) PCT Filed: Apr. 24, 2009
(86) PCT No.: PCT/AU09/00520

§ 371 Date: Jun. 7, 2011

(30) Foreign Application Priority Data
Feb. 24, 2008 (AU) 2008902052

(51) Int. Cl. A61K 33/00; A61P 17/00; A61P 31/00

(52) U.S. Cl. 424/600

(57) ABSTRACT

The present invention provides a disinfecting formulation useful, for example, for cleaning and disinfecting human or animal body parts, and in particular for disinfecting human hands. Typically, the formulation comprises alcohol, one or more essential oils comprising cineole, gelling agent and water. The invention also provides methods of disinfection of human and animal body parts and methods for preparing the formulation.

### FIELD OF THE INVENTION

The present invention provides a disinfecting formulation useful, for example, for cleaning and disinfecting human or animal body parts, and in particular for disinfecting human hands. The invention also provides methods of disinfection of human and animal body parts and methods for preparing the formulation.

### BACKGROUND OF THE INVENTION

There is an increasing need to develop improved methods and products for cleaning and disinfecting human and animal body parts, and in particular human hands. In addition to the normal need for individuals to maintain clean and disinfected hands in order to minimise the potential for transfer of pathogens, the members of a large number of professions are required to clean their hands during the course of their normal work. For example, those involved in the provision of human health care, in the preparation of food and beverage, the handling of animals, in child and geriatric care and in cleaning and waste management will all need to ensure their hands are regularly cleaned to avoid the transmission of pathogens that may cause disease either to themselves or to others. The conventional approach for hand cleaning is to use soap and water and in many workplaces where hygiene is paramount, liquid soaps or Alcohol Denat-based hand rubs (gels or foams) are adopted, many of which may include antimicrobial active agents such as isopropanol, chlorhexidine, triclorsan, quaternary ammonium compounds, iodinated compounds and the like. The problems with many of these antimicrobial agents are that they are harsh on the skin and many microbes have mutated to develop resistance to them. It is therefore desirable to develop fast acting hand cleaning formulations that exhibit high efficiency of killing or inactivating pathogens while being relatively gentle on the skin, to thereby allow for regular use (by not only trained professionals but also the general public) without development of skin irritation, inflammation, dryness, cracking, redness etc. as per an allergic response (particularly in the case of pre- and post-operative patients). Owing to some of the additives used in conventional hand cleaning formulations, it is necessary to also include a barrier cream and/or skin conditioner to protect and/or rehydrate the skin. It would be preferable if the additional use of such barrier creams and skin conditioners was not required. Ideally, hand cleaning formulations should neither dehydrate the skin nor cause skin irritation, inflammation, dryness, cracking, redness etc. as per an allergic response.

There are also a range of settings in which it is desirable to minimise the need for the use of water in conjunction with cleansing. In terms of daily hand cleansing this is a particular issue at the moment in parts of the world where climatic and rainfall conditions are changing and where water is becoming increasingly scarce. In parts of Australia, for example, availability of water is of increasing importance due to limits being placed at least in some areas on household water consumption. It is also desirable to have access to means of effectively cleansing for military applications or in the case of activities such as outdoor labour, camping, bushwalking and the like, where limited amounts of water may be available and where any available water will be for consumption rather than for washing.

It is in this context that the present inventors have conceived a disinfecting formulation that may be used in the absence of additional water. The inventors have adopted substantially natural products that exhibit surprising efficacy against a broad range of pathogenic microorganisms, and which can be used repeatedly on human and animal skin without any significant irritation, inflammation, dryness, cracking, redness etc. as per an allergic response. Primary ingredients of the formulations according to the invention include one or more essential oils comprising cineole, alcohol, a gelling agent and water.

German Patent Application No. 202007002978 discloses a gel composition comprising specified amounts of alcohol, thickener, at least one active agent selected from sedatives, healing promoters and/or anti-inflammatory agents, as well as water. For effective disinfection activity this formulation appears to require the presence of biguanide compounds, phenol compounds, iodine compounds or the like, which are not required for disinfection in the present invention. In preferred embodiments of the present invention such compounds are excluded from the formulation according to the present invention, such that disinfecting activity is contributed to by essential oils and alcohol as well as water, which surprisingly improves disinfection activity compared to formulations of essential oil and alcohol in the absence of water.

International Patent Publication No. WO 2005/084717 discloses a cleaning solution comprising ethanol, and essential oil with specified content of cineole. While the cleansing and disinfecting properties of alcohol such as ethanol and essential oils comprising cineole were understood, it was not expected that such agents could be combined into a formulation (such as a gel formulation), given that agents such as essential oils, ethanol, gelling agents such as waxes and gums, and water are generally not considered to be miscible. Furthermore, it was not expected that the incorporation of water into such a formulation would serve to improve activity against pathogenic microorganisms, in comparison to a formulation of essential oil and alcohol in the absence of water.

### SUMMARY OF THE INVENTION

In one embodiment the present invention relates to a disinfecting formulation comprising:
(a) alcohol;
(b) one or more essential oils comprising cineole;
(c) gelling agent; and
(d) water.

Preferably the formulation comprises one or more C₁ to C₁₀ alcohol, preferably one or more of methanol, ethanol and isopropanol. It is particularly preferred that the alcohol comprises ethanol of analytical grade (A.R).

Preferably the one or more essential oils is selected from eucalyptus, tea tree, bayleaf, spearmint and rosemary oils, preferably eucalyptus oil and most preferably eucalyptus oil of B.P. (British Pharmacopoeia) grade. In one preferred embodiment the formulation further comprises clove oil and/or sweet orange oil.

The gelling agent is preferably selected from one or more of vegetable, animal, mineral, petroleum or synthetic waxes, vegetable gums, starches, pectins, gelatine, chitin, chitosan, collagen, silica, cornstarch, glycols and carbomer (polyacrylic acid). For example, vegetable gums include locust bean gum, guar gum, xanthan gum, alginates, agar, carageenan, beta-glucan, gellan gum, gum arabic, gum tragacanth, karaya gum, locust bean gum, mastic gum, psyllium gum, spruce gum, ghatti gum and glucomannan and vegetable, animal, mineral, petroleum or synthetic waxes include beeswax, shellac wax, spermaceti, lanolin, bayberry wax, candelilla wax, carnauba wax, castor wax, esparto wax, jojoba oil, ouricury wax, rice bran wax, soy wax, ceresin waxes, montan wax, ozocerite, paraffin wax, microcrystalline wax, polyethylene waxes, chemically modified waxes, Fischer-Tropsch waxes, substituted amide waxes and polymerised α-olefins.

A particularly preferred gelling agent is “Anhydro Wax”.

In another preferred embodiment the ratio, by volume, of water to the essential oil is up to about 1.5:1.

In preferred embodiments the formulation comprises, by volume, from about 0.1% to about 5% gelling agent, from about 30% to about 85% alcohol and from about 5% to about 30% essential oil, preferably from about 0.5% to about 3% gelling agent, from about 60% to about 80% alcohol and from about 10% to about 25% essential oil and particularly preferably from about 1% to about 2% gelling agent, from about 70% to about 75% alcohol and from about 10% to about 15% essential oil.

In a preferred embodiment the formulation further comprises one or more emollient agents, such as one or more of lanolin, mineral, vegetable and synthetic oils and humectants. For example, humectants may comprise glycerine, propylene glycol, glyceryl triacetate, sorbitol, xylitol, melitol and polydextrose and vegetable oils may comprise coconut oil, jojoba oil, shea butter, mango butter and palm oil.

In one embodiment the formulation comprises, by volume, from about 1% to about 2% gelling agent, about 72% alcohol, about 11% essential oil, from about 1% to about 2% emollient agent and about 14% water.

In another embodiment of the invention there is provided a disinfecting formulation comprising, by volume:
(a) from about 65% to about 75% ethanol;
(b) from about 10% to about 15% eucalyptus oil;
(c) from about 0.5% to about 2% “Anhydro Wax”;
(d) from about 1% to about 4% glycerine; and
(e) water.

Preferably, the ratio, by volume, of water to the essential oil is up to about 1.5:1.

In a preferred embodiment the formulation comprises, by volume:
(a) about 72% ethanol;
(b) about 11% eucalyptus oil;
(c) from about 1% to about 2% “Anhydro Wax”;
(d) from about 1% to about 2% glycerine; and
(d) about 14% water.

In another preferred embodiment the formulation comprises, by volume:
(a) about 73% ethanol;
(b) about 10% eucalyptus oil;
(c) about 1.5% “Anhydro Wax”;
(d) about 4% glycerine; and
(d) about 11.5% water.

The invention also includes a method of disinfecting a human or animal body part comprising applying to the body part a formulation as defined above.

In one embodiment there is provided a method of disinfecting a human or animal body part comprising applying to the body part a formulation comprising, by volume:
(a) from about 65% to about 75% ethanol;
(b) from about 10% to about 15% eucalyptus oil;
(c) from about 0.5% to about 2% “Anhydro Wax”;
(d) from about 1% to about 4% glycerine; and
(e) water.

In a preferred embodiment the formulation used in the method comprises, by volume:
(a) about 72% ethanol;
(b) about 11% eucalyptus oil;
(c) from about 1% to about 2% “Anhydro Wax”;
(d) from about 1% to about 2% glycerine; and
(d) about 14% water.

In another preferred embodiment the formulation used in the method comprises, by volume:
(a) about 73% ethanol;
(b) about 10% eucalyptus oil;
(c) about 1.5% “Anhydro Wax”;
(d) about 4% glycerine; and
(d) about 11.5% water.

Preferably, the method is for disinfecting human hands.

In another embodiment of the invention there is provided a method of preparing a disinfecting formulation which comprises combining the “Anhydro Wax” and the eucalyptus oil to form a dispersion, which is then added to a mixture of the ethanol, glycerine and water with gentle mixing to produce the disinfecting formulation.

### DETAILED DESCRIPTION OT THE INVENTION

The reference to any prior art in this specification is not, and should not, be taken as an acknowledgment or any form of suggestion that the prior art forms part of the common general knowledge in Australia.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

In a preferred embodiment, the disinfecting formulation comprises alcohol such as a C₁ to C₁₀ alcohol, preferably selected from one or more of methanol, ethanol, and isopropanol. The alcohol should be non-toxic to animals and especially humans on the basis of dermal contact, since with normal use the formulation will come into contact with skin. It is preferred that the alcohol is ethanol of analytical grade (A.R). Preferably the formulation includes alcohol in an amount by volume of from about 30% to about 85%, preferably from about 60% to about 80%, more preferably from about 70% to about 75% and most preferably about 72% or about 73%.

The formulation comprises one or more essential oils and/or fractions thereof comprising cineole, which are generally obtained from distillation of fresh, dried or partially dried plants or plant derived materials. The essential oil may be obtained from components such as leaves, branches, shoots, stems, bark, seeds, fruit, roots, nuts or the like from one or more plants. Essential oil fractions may be obtained from distillation, purification, refining or the like of essential oils or components thereof. The essential oils and/or fractions are preferably selected from the group eucalyptus, tea tree, bayleaf, spearmint and rosemary oils, although other plant species may also give rise to essential oils containing cineole compounds, preferably 1,8-cineole, and preferably in an amount of 20% to 100% of the oil. In a still further preferred embodiment, eucalyptus oil of B.P. grade (where it complies with British Pharmacopoeia requirements) is used. Preferably the amount by weight of cineole in the essential oil, preferably eucalyptus oil, is at least about 60%, preferably from about 75% to about 85%. In a preferred embodiment, the amount by volume of the essential oil, preferably eucalyptus oil, within the formulation is from about 5% to about 30%, preferably from about 10% to 25%, more preferably from about 10% to about 15% and most preferably about 10% or about 11%.

The formulation will also include one or more gelling agents to impart certain characteristics to the formulation—that is to form a colloid that is to some extent immobilised and exhibits solid or semi-solid characteristics. Gelling agents that serve to thicken or impart a level of structural form to the formulation such as vegetable, animal, mineral, petroleum or synthetic waxes, vegetable gums, starches, pectins, gelatine, chitin, chitosan, collagen, silica, cornstarch, glycols and carbomer (polyacrylic acid) can be used. For example, vegetable gums include locust bean gum, guar gum, xanthan gum, alginates, agar, carageenan, beta-glucan, gellan gum, gum arabic, gum tragacanth, karaya gum, locust bean gum, mastic gum, psyllium gum, spruce gum, ghatti gum and glucomannan and vegetable, animal, mineral, petroleum or synthetic waxes include beeswax, shellac wax, spermaceti, lanolin, bayberry wax, candelilla wax, carnauba wax, castor wax, esparto wax, jojoba oil, ouricury wax, rice bran wax, soy wax, ceresin waxes, montan wax, ozocerite, paraffin wax, microcrystalline wax, polyethylene waxes, chemically modified waxes, Fischer-Tropsch waxes, substituted amide waxes and polymerised α-olefins. To be useful within the formulations of the present invention the gelling agents should be non-toxic in dermal use and substantially non-irritant and non-allergenic. The gelling agents may be present in amounts by volume of from about 0.1% to about 5%, preferably from about 0.5% to about 3% and more preferably from about 1% to about 2%.

Particularly preferred gelling agents are petroleum waxes, examples of which include paraffin wax and microcrystalline wax. A preferred petroleum wax is the proprietary formulation known as “Anhydro Wax”, which is commercially available from Intelisol Pty Ltd of 1/57 Malvern Street, Bayswater, Victoria, 3153, Australia (telephone no. +61 (0)3 9729 4260). If “Anhydro Wax” is adopted in the formulation it is preferably used in an amount by volume of from about 0.5% to about 2%, preferably from about 1% to about 2% or preferably about 1.5%.

The formulations of the invention also include water. Preferably, but not necessarily, the ratio, by volume, of water to the essential oil is up to about 1.5:1. That is, water will be included in the formulation in some amount and its preferable upper limit will be an amount, when measured by volume, no more than one and a half times the volume of essential oil present in the formulation. Typically, if a disinfecting formulation contains water, then the ratio, by volume, of water to essential oil is about 0.5:1. Thus, it was unexpected that a ratio of up to about 1.5:1 could be used in the present invention to prepare homogeneous formulations. Preferably the water will be purified to remove pathogens such as by microfiltration and/or distillation.

Although not essential the formulation will preferably also include one or more emollient agents that will serve to soften the skin, usually by improving skin hydration (i.e. moisturizing the skin). Examples of suitable emollients are lanolin, mineral, vegetable and synthetic oils and humectants. Humectants are hygroscopic agents that have the ability to form hydrogen bonds and attract water to thereby have a moisturizing effect. For example, humectants may comprise glycerine, propylene glycol, glyceryl triacetate, sorbitol, xylitol, melitol and polydextrose. Examples of vegetable oils include coconut oil, jojoba oil, shea butter, mango butter and palm oil. The emollients utilised should all be non-toxic in dermal use and substantially non-irritant and non-allergenic.

Preferably the emollient agent, if included in the formulation, will be present in an amount by volume of from about 0.1% to about 5%, preferably from about 0.5% to about 3% and more preferably from about 1% to about 2%. In a particularly preferred embodiment the emollient agent is glycerine, preferably vegetable glycerine which is readily commercially available, which may for example be present in an amount by volume of from about 1% to about 4%, preferably from about 1% to about 2% or preferably about 4%. It should be understood, however, that depending upon the nature of the gelling agent selected a single agent or a group of agents may together form the functions of both the gelling and emollient components. In the case of a single component filling both functions it could be present in the formulation in an amount of from about 1% to about 10%, preferably from about 2% to about 8%, more preferably from about 3% to about 5% by volume.

In another preferred embodiment of the invention additional ingredients not otherwise specified may be included within the formulation such as for example essential oils that do not include cineole to any significant extent (e.g. clove oil, sweet orange oil), other agents active against microorganisms, aromatic scents, stabilisers and the like that are routinely used in dermal preparations, which should all be non-toxic in dermal use and substantially non-irritant and non-allergenic.

The formulation is a disinfectant in that it has anti-microbial activity and hence will kill, slow growth and/or cellular division of microorganisms and other pathogens such as bacteria (Gram positive and Gram negative), viruses, fungi, protozoa, mites, algae, nematodes and the like (collectively referred to as “flora”). By application of the solution to the skin preferably at least 90%, more preferably at least 95%, particularly preferably at least 98% or 99% and most preferably at least 99.5% or 99.9% of flora on the skin will be killed or otherwise inactivated. The superior disinfecting nature of the preferred formulation of the invention (comprising ethanol, eucalyptus oil, “Anhydro Wax”, glycerine and water) is attributed to the prolonged presence of ethanol on the skin. That is, upon application of the formulation, the eucalyptus oil and glycerine components delay evaporation of the ethanol component, thereby allowing the ethanol component to kill any flora present on the skin and prevent regrowth for an extended period of time. Conventional disinfecting formulations contain hazardous chemicals (e.g., chlorhexidine glutamate) to achieve such persistent anti-microbial activity.

The formulation will be physiologically compatible with skin. In addition to disinfecting, the formulation may assist to clean skin (by removing dead skin cells, grease, grime and the like) and/or manage minor wounds and skin disorders (e.g., cuts, scratches, abrasions, eczema, dermatitis). The formulation may be dispensed from a pump container, canister or tube, or alternatively a single dose of the formulation may be provided in a sealed foil or laminated plastic (polypropylene) sachet. In use an amount sufficient to coat the hands or skin to be cleansed will be applied to the skin. The formulation is a “leave on” application and dries naturally. However, excess formulation may be removed by wiping with paper towel or a dry cloth (which should be sterile in the case of medical/surgical applications).

The disinfecting formulation of the invention can be prepared by combining the gelling agent and the essential oil comprising cineole to form a dispersion, which is then added to a mixture of the alcohol, emollient agent (if present) and water, preferably slowly and with gentle mixing to ensure homogeneity but without aeration, as it is preferred to avoid the inclusion of air bubbles within the formulation. For example the mixing process may be conducted using a vacuum mixing technique in order to minimise aeration.

A preferred formulation of the invention may be prepared by combining “Anhydro Wax” and eucalyptus oil to form a dispersion, which is then added to a mixture of ethanol, glycerine and water with gentle mixing. The various components may be added in amounts as referred to above.

The present invention will now be described further with reference to the following non-limiting examples:

### EXAMPLES

### Example 1

### Preparation of Disinfecting Gel Formulation

A formulation was prepared containing the ingredients listed in Table 1 below in the percentage amounts by volume as indicated.

TABLE 1

72% A.R. grade ethanol
11% B.P. grade eucalyptus oil
1.5% “Anhydro Wax”
1.5% glycerine
14% distilled water

The gel was prepared by combining the “Anhydro Wax” and eucalyptus oil to form a dispersion, which was then added to a mixture of the ethanol, glycerine and water with gentle mixing over one hour.

### Example 2

### Anti-viral Activity Testing (Herpes Simplex)

### Objective

To determine whether a disinfectant product, antiseptic hand gel, was veridical against Herpes Simplex Virus Type 1 in a suspension test, using accepted criteria for making veridical claims.

### Materials and Methods

### A. Virus Strain

The test virus used was Herpes virus Type 1 obtained from ATCC. The virus was stored in liquid nitrogen prior to use.

### B. Cell Substrate

The Vero cells were obtained from ATCC. The Vero cells were stored in liquid nitrogen prior to use. Cells were thawed and sub-cultured in DMEM cell growth medium.

### C. Test Products

Product prepared according to Example 1 was tested. The sample was assigned the laboratory reference number 0802451. Sample was tested neat.

### D. Experimental Design

The design may be summarized as consisting of application of test virus into the test product.

Any surviving virus in test or control conditions was assayed using Vero cells and the observation of Virus Cytopathic Effect (CPE).

### E. Reagents and Suppliers

1. Medium DMEM, supplied by Sigma
2. L-glutamine, supplied by Sigma
3. Foetal Bovine Serum (FBS), supplied by Sigma
4. Trypsin/Versene (TEDTA), supplied by Sigma
5. Flat-bottom micro titration plates, supplied by Crown Scientific
6. Hepes Buffer supplied by Sigma
7. Phosphate Buffer Solution (PBS), supplied by ams Labs.

### F. Preparation of Cell Substrate

1. All work was carried out in a Biohazard cabinet.
2. The Vero cell growth media was prepared by aseptically combining 5 ml of Foetal bovine Serum to 100 ml of Medium DMEM.
3. The contents of a Vero cell flask were decanted into a 500 ml discard jar, and using aseptic technique, approximately 2 ml of TEDTA was introduced into the flask and the monolayer was thoroughly washed with TEDTA. This was then discarded and the process was repeated.
4. The flask was incubated at 37° C. for approximately 3 minutes, with the flask checked every minute to see whether the cells were lifting from the plastic. Progress was checked using an inverted microscope.
5. When all the cells were detached, 40 ml of growth media was added and the flask was shaken gently to suspend the cells in the media.
6. The contents of the Flask were aseptically poured into the base of a sterile petri dish.
7. Using a multi-channel pipette, 100 μl of cell suspension was dispensed into each well.
8. The plates were incubated in the CO₂ incubator with an atmosphere of 5% CO₂ in air at a temperature of 37° C.±1° C. for 24 hours.

### G. Preparation of Sephadex Gel Column

1. A slurry of Sephadex Gel was aseptically prepared by combining 100 ml of Sterile Distilled Water with 5 g of Sephadex powder.
2. This mixture was left at 4° C. overnight before being sterilised at 121° C. for 15 minutes in autoclave.
3. 3 ml sterile gel was aseptically removed and was placed into the barrel of 5 ml syringe. Gel was equilibrated with PBS and drained before applying test sample.

### H. Conduct of the Virus/Disinfectant Test

1. The virus was removed from the liquid nitrogen and thawed at 37° C.±2° C. 0.2 ml of virus suspension was added into 1.8 ml of each sample preparation for the 5 and 10 minute contact times.
2. At 5 and 10 minutes, 0.6 ml of the sample was then added to the gel column. This eluate was collected and regarded as 1:10 dilution. Further serial dilution to 10⁻⁷ was then made in the maintenance medium.

### I. Preparation of Virucidal Assay Controls

1. The positive control was prepared by adding 0.2 ml of virus suspension to 1.8 ml of maintenance media, further serial dilutions were carried out the same way as per product assay.
2. 0.1 ml of 10⁻² dilution of positive virus control was spiked into 0.9 ml of 10⁻², 10⁻³, dilutions of the product cytotoxicity control. This material was then assayed for virus.

### J. Preparation Of Cxtotoxicity Control

0.2 ml of maintenance media was added to 1.8 ml of each sample preparation.
0.6 ml of this mixture was passed through gel column. Eluate was collected and further 10⁻² and 10⁻³ dilution was then made in the maintenance medium.

### K. Preparation of Neutralisation Control

0.1 ml of 10⁻³ dilution of virus control was added to 0.9 ml of 10⁻³ and 10⁻⁴ dilution of cytotoxicity control.

### L. Virus Assay

1. Confluent Vero monolayers in 96 well plates were obtained by decanting the culture supernatant into the microtitre plate media discard tray.
2. The plates were washed once with PBS. Commencing with the highest dilution of the test samples, 100 μl of each dilution were dispensed into the designated four wells. After one hour incubation at 37±2° C. in humidified CO₂ incubator, plates were washed once with PBS and 200 μl of the maintenance medium was added to each well. This plating procedure was followed for all test and control materials and all assay controls. When all wells were filled, the lid of the 96 well microtitre plate was replaced and the plate incubated in the 5% CO₂ humidified incubator at 37±2° C. for 6 days.

### M. Reading Virus Titration Results

1. After the incubation period, the plates were microscopically examined for Virus Cytopathic Effect (CPE).
2. The positive wells and negative wells at each dilution were recorded on the Virus Titration Worksheet.

### N. Calculation of Virus Titre

The Reed & Muench LD50 Method is used for determining the virus titre endpoint.

### Results

The untreated Herpes control had a log₁₀ titre of 5.5 (see Table 2).

The virus used in the present study was completely inactivated by the test product at the contact times of 5 and 10 minutes at room temperature (see Table 3 and Table 4).

Sample showed cytotoxicity at the 10⁻² dilution (Table 5).

Product showed signs of complete neutralisation at the 10⁻³ dilution (Table 6).

TABLE 2
VIRUS CONTROL RESULTS

| | | |
|---|---|---|
| Virus | Number | |
| Dilution | Inoculated | Individual Responses |

| | | | | | |
|---|---|---|---|---|---|
| 10⁻² | 4 | + | + | + | + |
| 10⁻³ | 4 | + | + | + | + |
| 10⁻⁴ | 4 | + | + | + | + |
| 10⁻⁵ | 4 | + | + | + | + |
| 10⁻⁶ | 4 | − | − | − | − |
| 10⁻⁷ | 4 | − | − | − | − |

| | |
|---|---|
| Total hosts: | 24 |

Calculated virus titre = 10^{5.5} TCID₅₀(5.5 log₁₀)
Note:
Presence of virus in each response is recorded as “+”
Absence virus in each response is recorded as “−”
Cytotoxic response is recorded as “C”

TABLE 3
RESULTS FOR VIRUS TREATED WITH PRODUCT

| Virus | Contact time 5 MINUTES | |
|---|---|---|
| Dilution | No. Innoc. | Individual Responses |

| | | | | | |
|---|---|---|---|---|---|
| 10⁻² | 4 | C | C | C | C |
| 10⁻³ | 4 | − | − | − | − |
| 10⁻⁴ | 4 | − | − | − | − |
| 10⁻⁵ | 4 | − | − | − | − |
| 10⁻⁶ | 4 | − | − | − | − |

| | |
|---|---|
| Total hosts: | 20 |

Calculated virus titre = 10^{2.5} TCID₅₀ (2.5 log₁₀)

TABLE 4
RESULTS FOR VIRUS TREATED WITH PRODUCT

| Virus | Contact time 10 MINUTES | |
|---|---|---|
| Dilution | No. Innoc. | Individual Responses |

| | | | | | |
|---|---|---|---|---|---|
| 10⁻² | 4 | C | C | C | C |
| 10⁻³ | 4 | − | − | − | − |
| 10⁻⁴ | 4 | − | − | − | − |
| 10⁻⁵ | 4 | − | − | − | − |
| 10⁻⁶ | 4 | − | − | − | − |

| | |
|---|---|
| Total hosts: | 20 |

Calculated virus titre = 10^{2.5} TCID₅₀ (2.5 log₁₀)

TABLE 5
RESULTS FOR CYTOTOXICITY CHECK

| | | |
|---|---|---|
| Virus | | |
| Dilution | No. Innoc. | Individual Responses |

| | | | | | |
|---|---|---|---|---|---|
| 10⁻² | 4 | C | C | C | C |
| 10⁻³ | 4 | − | − | − | − |

| | |
|---|---|
| Total hosts: | 8 |

Calculated virus titre = 10^{2.5} TCID₅₀ (2.5 log₁₀)
Note:
+ represents infected hosts showing haemagglutination.
− represents infected hosts showing no haemagglutination
C presence of cytotoxicity response is recorded as “C”

TABLE 6
RESULTS FOR PRODUCT NEUTRALIZATION

| | | |
|---|---|---|
| Virus | | |
| Dilution | No. Innoc. | Individual Responses |

| | | | | | |
|---|---|---|---|---|---|
| 10⁻² | 4 | C | C | C | C |
| 10⁻³ | 4 | + | + | + | + |

| | |
|---|---|
| Total hosts: | 8 |

TABLE 7
LOG₁₀ REDUCTION OF VIRUS AFTER TREATMENT

| | Titre | Reduction |
|---|---|---|
| Treatment | (Log₁₀₎ | (Log₁₀) |
| Virus Control | 5.5 | — |
| 5 mins Treatment | 2.5 | 3.0 |
| 10 mins Treatment | 2.5 | 3.0 |

### Conclusions

This study clearly demonstrates that the test product at neat concentration was able to kill Herpes simplex type 1 virus at room temperature with contact times of 5 and 10 minutes in a suspension test model (Table 7). Evidence of complete viral neutralisation after 5 and 10 minute exposure periods, with a 3.0 log reduction in viral titre, indicates that the test 5 and 10 minute contact times showed compliance with the efficacy requirement for disinfectant as specified in TGO 54 and 54A.

### Example 3

### Anti-Viral Activity Testing (Adeno Virus)

Using the same protocol adopted in Example 2, but instead Adeno type 2 virus obtained from the ATCC antiviral activity of the gel of Example 1 was tested. In this case MRCS cells obtained from the ATCC were used as the cell substrate. The cells were thawed and sub-cultured in EMEM cell growth medium.

### Results

The untreated Adeno Type 2 virus control had a log₁₀ titre of 5.5 (see Table 8).

The virus used in the present study was completely inactivated by the test procedure at the contact times of 5 and 10 minutes at room temperature (see Table 9 and Table 10).

Sample showed cytotoxicity at the 10⁻² dilution (Table 11).

Product showed signs of complete neutralisation at the 10⁻³ dilution (Table 12).

TABLE 8
VIRUS CONTROL RESULTS

| | | |
|---|---|---|
| Virus | Number | |
| Dilution | Inoculated | Individual Responses |

| | | | | | |
|---|---|---|---|---|---|
| 10⁻² | 4 | + | + | + | + |
| 10⁻³ | 4 | + | + | + | + |
| 10⁻⁴ | 4 | + | + | + | + |
| 10⁻⁵ | 4 | + | + | + | + |
| 10⁻⁶ | 4 | − | − | − | − |
| 10⁻⁷ | 4 | − | − | − | − |

| | |
|---|---|
| Total hosts: | 24 |

Calculated virus titre = 10^{5.5} TCID₅₀(5.5 log₁₀)
Note:
Presence of virus in each response is recorded as “+”
Absence virus in each response is recorded as “−”
Cytotoxic response is recorded as “C”

TABLE 9
RESULTS FOR VIRUS TREATED WITH PRODUCT

| Virus | Contact time 5 MINUTES | |
|---|---|---|
| Dilution | No. Innoc. | Individual Responses |

| | | | | | |
|---|---|---|---|---|---|
| 10⁻² | 4 | C | C | C | C |
| 10⁻³ | 4 | − | − | − | − |
| 10⁻⁴ | 4 | − | − | − | − |
| 10⁻⁵ | 4 | − | − | − | − |
| 10⁻⁶ | 4 | − | − | − | − |

| | |
|---|---|
| Total hosts: | 20 |

Calculated virus titre = 10^{2.5} TCID₅₀ (2.5 log₁₀)

TABLE 10
RESULTS FOR VIRUS TREATED WITH PRODUCT

| Virus | Contact time 10 MINUTES | |
|---|---|---|
| Dilution | No. Innoc. | Individual Responses |

| | | | | | |
|---|---|---|---|---|---|
| 10⁻² | 4 | C | C | C | C |
| 10⁻³ | 4 | − | − | − | − |
| 10⁻⁴ | 4 | − | − | − | − |
| 10⁻⁵ | 4 | − | − | − | − |
| 10⁻⁶ | 4 | − | − | − | − |

| | |
|---|---|
| Total hosts: | 20 |

Calculated virus titre = 10^{2.5} TCID₅₀ (2.5 log₁₀)

TABLE 11
RESULTS FOR CYTOTOXICITY CHECK

| | | |
|---|---|---|
| Virus | | |
| Dilution | No. Innoc. | Individual Responses |

| | | | | | |
|---|---|---|---|---|---|
| 10⁻² | 4 | C | C | C | C |
| 10⁻³ | 4 | − | − | − | − |

| | |
|---|---|
| Total hosts: | 8 |

Calculated virus titre = 10^{2.5} TCID₅₀ (2.5 log₁₀)
Note:
+ represents infected hosts showing haemagglutination.
− represents infected hosts showing no haemagglutination
C presence of cytotoxicity response is recorded as “C”

TABLE 12
RESULTS FOR PRODUCT NEUTRALIZATION

| | | |
|---|---|---|
| Virus | | |
| Dilution | No. Innoc. | Individual Responses |

| | | | | | |
|---|---|---|---|---|---|
| 10⁻² | 4 | C | C | C | C |
| 10⁻³ | 4 | + | + | + | + |

| | |
|---|---|
| Total hosts: | 8 |

TABLE 13
LOG₁₀ REDUCTION OF VIRUS AFTER TREATMENT

| | Titre | Reduction |
|---|---|---|
| Treatment | (Log₁₀) | (Log₁₀) |
| Virus Control | 5.5 | — |
| 5 mins Treatment | 2.5 | 3.0 |
| 10 mins Treatment | 2.5 | 3.0 |

### Conclusions

This study clearly demonstrates that, the test product at neat concentration, was able to kill Adeno type 2 virus at room temperature with contact times of 5 and 10 minutes in a suspension test model (Table 13). Evidence of viral neutralisation after 5 and 10 minute exposure periods, with a 3.0 log reduction in viral titre, indicates that the test 5 and 10 minute contact times showed compliance with the efficacy requirement for disinfectant as specified in TGO 54 and 54A.

### Example 4

### Anti-Viral Activity Testing (Human Influenza Virus)

### Objective

To determine whether the gel formulation of Example 1 was veridical against Human Influenza Virus Type A in a suspension test, using accepted criteria for making veridical claims.

### Materials and Methods

### A. Virus Strain

The test virus used in this study was Human Influenza Virus Type A (strain PR8) obtained from ICPMR.

### B. Cell Substrate

The MDCK cells were obtained from CSL Bioscience. The MDCK cells were stored in liquid nitrogen prior to use. Cells were thawed and sub-cultured in DMEM cell growth medium.

### C. Test Products

Product prepared according to Example 1 was tested. The sample was assigned the laboratory reference number 0805139. Sample was tested neat.

### D. Experimental Design

The design may be summarized as consisting of application of test virus into the test product.

Any surviving virus in test or control conditions was assayed using Vero cells and the observation of CPE.

### E. Reagents and Suppliers

1. PBS was used for titrating haemagglutinating activity. It was supplied as pre-formulated tablets by Oxoid Australia Pty Ltd and made up as per manufacturer's instructions. 0.5% FBS was added before use.
   2. Chicken red blood cells were supplied by IMVS Veterinary services in Alsever's Solution prepared by ams Labs. They were washed three times in PBS and adjusted to 0.8% v/v before use.
3. Medium EMEM plus all the supplements needed to prepare maintenance medium was supplied by Sigma Aldrich.

### F. Preparation of Cell Substrate

1. All work was carried out in a Biohazard cabinet.
2. The MDCK cell cultures were grown in EMEM growth medium.
3. The contents of a MDCK flask were decanted into a 500 ml discard jar, and using aseptic technique, approximately 2 ml of TEDTA was introduced into the MDCK flask. The flask was gently rotated to ensure that all the surface of the monolayer was covered with the TEDTA.
4. The flask was incubated at 37° C. for approximately 30 minutes, with the flask checked every few minutes to see whether the cells were lifting from the plastic. Progress was checked using an inverted microscope.
5. When all the cells were detached, 40 ml of MDCK growth medium was added and the flask was shaken gently to suspend the cells in the medium.
6. Equal volumes of the cell suspension were transferred into two sterile McCartney bottles.
7. Using a multi-channel pipette, 100 μl of cell suspension was dispensed into each well,
8. The plates were incubated in the CO₂ incubator with an atmosphere of 5% CO₂ in air at a temperature of 37° C.±2° C. for 24 hours.

### G. Preparation of Sephadex Gel Column

1. A slurry of Sephadex Gel was aseptically prepared by combining 100 ml of Sterile Distilled Water with 5 g of Sephadex powder.
2. This mixture was left at 4° C. overnight before being sterilised at 121° C. for 15 minutes in autoclave.
3. 3 ml sterile gel was aseptically removed and was placed into the barrel of 5 ml syringe. Gel was equilibrated with PBS and drained before applying test sample.

### H. Conduct of the Virus/Disinfectant Test

1. The virus was removed from the liquid nitrogen and thawed at 37° C.±2° C. 0.2 ml of virus suspension was added into 1.8 ml of each sample preparation for the 1 and 3 minute contact times,
2. At 1 and 3 minutes, 0.6 ml of the sample was then added to the gel column. This eluate was collected and regarded as 1:10 dilution. Further serial dilution to 10⁻⁷ was then made in the maintenance medium,

### I. Preparation of Virucidal Assay Controls

The positive control was prepared by adding 0.2 ml of virus suspension to 1.8 ml of maintenance media, further serial dilutions were carried out the same way as per product assay.

### J. Preparation of Cxtotoxicity Control

0.2 ml of maintenance media was added to 1.8 ml of each sample preparation. 0.6 ml of this mixture was passed through gel column. Eluate was collected and further 10⁻² and 10⁻³ dilution was then made in the maintenance medium.

### K. Preparation of Neutralisation Control

0.1 ml of 10⁻³ dilution of virus control was added to 0.9 ml of 10⁻² and 10⁻³ dilution of cytotoxicity control.

### L. Virus Assay

1. Confluent MDCK monolayers in 96 well plates were obtained by decanting the culture supernatant into the microtitre plate media discard tray.
2. The plates were washed once with PBS. Commencing with the highest dilution of the test samples, 100 μl of each dilution were dispensed into the designated four wells. After one hour incubation at 37±2° C. in humidified CO₂ incubator, plates were washed once with PBS and 200 μl of the maintenance medium was added to each well. This plating procedure was followed for all test and control materials. When all wells were filled, the lid of the 96 well microtitre plate was replaced and the plate incubated in the 5% CO₂ humidified incubator at 37±2° C. for 6 days.
3. Any surviving virus in test was assayed using haemagglutination assay.
4. To examine for haemagglutinin activity, a further 0.1 mL of 0.8% washed chicken red blood cells were added to each well containing 0.1 mL test supernatant. The plates were gently agitated to mix the red blood cells and left to stand at ambient room temperature for 45 minutes.

### M. Reading Virus Titration Results

Each well was scored for absence of haemagglutination, by observation of a “button” of red blood cells on the bottom of the well (−), or presence of haemagglutination, by observation of a uniformly distributed layer of red cells over the bottom of the plate (+), and cytotoxicity to MDCK cells, by observation of no viral growth (C). Presence of haemagglutination was taken as evidence of virus replication in the host and recorded accordingly. The positive and negative wells at each dilution were recorded.

### N. Calculation of Virus Titre

The Reed & Muench LD50 Method is used for determining the virus titre endpoint.

### Results

The untreated human influenza A (PR8) virus control had a log₁₀ titre of 5.7 (see Table 14).

The virus used in the present study was completely inactivated by the test product at the contact times of 1 and 3 minutes at room temperature (see Table 15 and Table 16).

Sample showed no cytotoxicity at the 10⁻² dilution (Table 17).

Product showed signs of complete neutralisation at the 10⁻² dilution (Table 18).

The washed 0.8% chicken red blood cells settled and formed normal “buttons” in the absence of virus.

TABLE 14
VIRUS CONTROL RESULTS

| Virus Dilution | No. Inoc. | Individual Responses | | | | | |
|---|---|---|---|---|---|---|---|
| 10⁻² | 6 | + | + | + | + | + | + |
| 10⁻³ | 6 | + | + | + | + | + | + |
| 10⁻⁴ | 6 | + | + | + | + | + | + |
| 10⁻⁵ | 6 | + | + | + | + | + | + |
| 10⁻⁶ | 6 | + | − | − | − | − | − |
| 10⁻⁷ | 6 | − | − | − | − | − | − |

| | |
|---|---|
| Total hosts: | 36 |

Calculated virus titre = 10^{5.7}TCID₅₀(5.7 log₁₀)
Note:
Presence of virus in each response is recorded as “+”
Absence virus in each response is recorded as “−”
Cytotoxic response is recorded as “C”

TABLE 15
RESULTS FOR VIRUS TREATED WITH PRODUCT

| | Contact time 1 MINUTE | | | | | | |
|---|---|---|---|---|---|---|---|
| Virus Dilution | No. Inoc. | Individual Responses | | | | | |
| 10⁻² | 6 | − | − | − | − | − | − |
| 10⁻³ | 6 | − | − | − | − | − | − |
| 10⁻⁴ | 6 | − | − | − | − | − | − |
| 10⁻⁵ | 6 | − | − | − | − | − | − |
| 10⁻⁶ | 6 | − | − | − | − | − | − |
| Total hosts: | 30 | | | | | | |

Calculated virus titre <10^{1.5}TCID₅₀ (<1.5 log₁₀)

TABLE 16
RESULTS FOR VIRUS TREATED WITH PRODUCT

| Virus | Contact time 3 MINUTES | | | | | | |
|---|---|---|---|---|---|---|---|
| Dilution | No. Inoc. | Individual Responses | | | | | |
| 10⁻² | 6 | − | − | − | − | − | − |
| 10⁻³ | 6 | − | − | − | − | − | − |
| 10⁻⁴ | 6 | − | − | − | − | − | − |
| 10⁻⁵ | 6 | − | − | − | − | − | − |
| 10⁻⁶ | 6 | − | − | − | − | − | − |
| Total hosts: | 30 | | | | | | |

Calculated virus titre <10^{1.5}TCID₅₀(<1.5 log₁₀)

TABLE 17
RESULTS FOR CYTOTOXICITY CHECK

| Virus | | | | | | | |
|---|---|---|---|---|---|---|---|
| Dilution | No. Inoc. | Individual Responses | | | | | |
| 10⁻² | 6 | − | − | − | − | − | − |
| 10⁻³ | 6 | − | − | − | − | − | − |
| Total hosts: | 12 | | | | | | |

Calculated virus titre <10^{1.5}TCID₅₀(<1.5 log₁₀)

TABLE 18
RESULTS FOR PRODUCT NEUTRALIZATION

| Virus | | | | | | | |
|---|---|---|---|---|---|---|---|
| Dilution | No. Inoc. | Individual Responses | | | | | |
| 10⁻² | 6 | + | + | + | + | + | + |
| 10⁻³ | 6 | + | + | + | + | + | + |
| Total hosts: | 12 | | | | | | |

Note:
+ represents infected hosts showing haemagglutination
− represents infected hosts showing no haemagglutination
C represents cytotoxic response

TABLE 19
LOG₁₀ REDUCTION OF VIRUS AFTER TREATMENT

| | Titre | Reduction |
|---|---|---|
| Treatment | (Log₁₀) | (Log₁₀) |
| Virus Control | 5.7 | — |
| 1 min Treatment | <1.5 | >4.2 |
| 3 mins Treatment | <1.5 | >4.2 |

### Conclusions

This study clearly demonstrates that the test product at neat concentration was able to kill human influenza A completely at room temperature with contact times of 1 and 3 minutes in a suspension test model (Table 19), Evidence of viral neutralisation after 1 and 3 minute exposure periods, with a greater than 4.2 log reduction in viral titre, indicates that the test at 1 and 3 minute contact times showed veridical properties.

### Example 5

### Anti-microbial Activity Challenge Test (TM110)

### Objective

To determine whether the gel formulation of Example 1 demonstrated anti-microbial activity against Methicillin-resistant Staphylococcus aureus and Vancomycin-resistant Enterococcus faecalis.

### Conditions

Test organisms: 1. Methicillin-resistant Staphylococcus aureus (MRSA)
2. Vancomycin-resistant Enterococcus faecalis (VRE)

### Test Concentration Neat

Contact Times: 30 seconds and 1 minute

### Test Temperature: Ambient

### Results

TABLE 20
Surviving MRSA after exposure to gel formulation

| | Surviving organisms (CFU/mL) | | | |
|---|---|---|---|---|
| | 30 sec | | 1 min | |
| | CFU/mL | Log | CFU/mL | Log |
| Sample Details | (log₁₀) | Reduction | (log₁₀) | Reduction |
| SunnyWipes Gel | <10 | >4.8 | <10 | >4.8 |
| | (<1) | | (<1) | |

| | |
|---|---|
| Inoculum | 6.3 × 10⁵ (5.8 Logs) |

CFU = Colony Forming Unit

TABLE 21
Surviving VRE after exposure to gel formulation

| | Surviving organisms (CFU/mL) | | | |
|---|---|---|---|---|
| | 30 sec | | 1 min | |
| | CFU/mL | Log | CFU/mL | Log |
| Sample Details | (log₁₀) | Reduction | (log₁₀) | Reduction |
| SunnyWipes Gel | <10 | >4.8 | <10 | >4.8 |
| | (<1) | | (<1) | |

| | |
|---|---|
| Inoculum | 6.0 × 10⁵ (5.8 Logs) |

CFU = Colony Forming Unit

### Conclusions

The sample successfully demonstrated anti-microbial activity against Methicillin-resistant Staphylococcus aureus and Vancomycin-resistant Enterococcus faecalis by more than 4.8 log reduction (kill >99.998%) after 30 seconds and 1 minute of contact time, when tested neat in conditions described above.

### Example 6

### Anti-microbial Activity Challenge Test (EN 1040:2006)

### Objective

To determine whether the gel formulation of Example 1 demonstrated bactericidal activity according to the EN 1040:2006 standard.

### Conditions

Test organisms: 1. Staphylococcus aureus ATCC 6538
2. Pseudomonas aeruginosa ATCC 15442

### Inoculum Level Approx. 1−5×10⁸ Colony Forming Units (CFU)/mL

### Test Concentration Neat

Contact Time: 5 minutes

### Test Temperature: Ambient

### Results

TABLE 22
Surviving organisms after 5 minutes exposure to gel formulation

| | Surviving organisms (CFU/mL) and Log Reduction | | | |
|---|---|---|---|---|
| | S. aureus | | Ps. aeruginosa | |
| | CFU/mL | Log | CFU/mL | Log |
| Sample | (log₁₀) | Reduction | (log₁₀) | Reduction |
| Antiseptic | <100 | >5.66 | <100 | >5.76 |
| Gel | (<2.15) | | (<2.15) | |

| | | |
|---|---|---|
| In test Inoculum | 6.5 × 10⁷ | 8.2 × 10⁷ |
| | (7.81) | (7.91) |

Notes:
1. Product Neutralisation—The validated neutraliser was T6 (a mixture of Tryptone Soy Broth, Lecithin and Tween 80) for both organisms tested.
2. All controls and validation were satisfactory.

### Conclusions

The sample successfully demonstrated bactericidal activity according to the EN 1040:2006 standard. The sample showed more than 5.66 log reduction against Staphylococcus aureus ATCC 6538 and more than 5.76 log reduction against Pseudomonas aeruginosa ATCC 15442 when tested neat in conditions described above. The sample requires 5 minutes of contact time to meet the accepted criteria for making bactericidal claims according to those required by the EN 1040:2006 standard.

### Example 7

### Anti-Microbial Activity Challenge Test (prEN 12054)

### Objective

To determine whether the gel formulation of Example 1 demonstrated bactericidal activity according to the prEN 12054 standard.

### Conditions

Test organisms: 1. Pseudomonas aeruginosa ATCC 15442
2. Escherichia coli NCTC 10538
3. Staphylococcus aureus ATCC 6538
4. Enterococcus hirae ATCC 10541

### Inoculum Level: Approx. 1−3×10⁸ Colony Forming Units (CFU)/mL

### Test Concentration Neat

Contact Times: 30 seconds and 1 minute

### Test Temperature Ambient

TABLE 23
Surviving Pseudomonas aeruginosa after 30 seconds
and 1 minute exposure to gel formulation

| | Surviving organisms (CFU/mL) and Log Reduction | | | |
|---|---|---|---|---|
| | 30 seconds | | 1 minute | |
| | CFU/mL | Log | CFU/mL | Log |
| Sample | (log₁₀) | Reduction | (log₁₀) | Reduction |
| Antiseptic | <3.0 × 10² | >5.84 | <3.0 × 10² | >5.84 |
| Hand Gel | (<2.48) | | (<2.48) | |

| | |
|---|---|
| In test Inoculum | 2.11 × 10⁸ |
| | (8.32) |

TABLE 24
Surviving Escherichia coli after 30 seconds
and 1 minute exposure to gel formulation

| | Surviving organisms (CFU/mL) and Log Reduction | | | |
|---|---|---|---|---|
| | 30 seconds | | 1 minute | |
| | CFU/mL | Log | CFU/mL | |
| Sample | (log₁₀) | Reduction | (log₁₀) | Log Reduction |
| Antiseptic | <3.0 × 10² | >5.88 | <3.0 × 10² | >5.88 |
| Hand Gel | (<2.48) | | (<2.48) | |

| | |
|---|---|
| In test Inoculum | 2.31 × 10⁸ |
| | (8.36) |

TABLE 25
Surviving Staphylococcus aureus after 30 seconds
and 1 minute exposure to gel formulation

| | Surviving organisms (CFU/mL) and Log Reduction | | | |
|---|---|---|---|---|
| | 30 seconds | | 1 minute | |
| | CFU/mL | Log | CFU/mL | Log |
| Sample | (log₁₀) | Reduction | (log₁₀) | Reduction |
| Antiseptic | <3.0 × 10² | >5.97 | <3.0 × 10² | >5.97 |
| Hand Gel | (<2.48) | | (<2.48) | |

| | |
|---|---|
| In test Inoculum | 2.83 × 10⁸ |
| | (8.45) |

TABLE 26
Surviving Enterococcus hirae after 30 seconds
and 1 minute exposure to gel formulation

| | Surviving organisms (CFU/mL) and Log Reduction | | | |
|---|---|---|---|---|
| | 30 seconds | | 1 minute | |
| | CFU/mL | Log | CFU/mL | Log |
| Sample | (log₁₀) | Reduction | (log₁₀) | Reduction |
| Antiseptic | <3.0 × 10² | >5.91 | <3.0 × 10² | >5.91 |
| Hand Gel | (<2.48) | | (<2.48) | |

| | |
|---|---|
| In test Inoculum | 2.46 × 10⁸ |
| | (8.39) |

### Conclusions

The sample successfully demonstrated bactericidal activity according to the prEN 12054 standard. The sample showed more than 5 log reduction against Pseudomonas aeruginosa ATCC 15442, Escherichia coli NCTC 10538, Staphylococcus aureus ATCC 6538 and Enterococcus hirae ATCC 10541 when tested neat in conditions described above. The sample requires at least 30 seconds of contact time to meet the accepted criteria for making bactericidal claims according to those required by the prEN 12054 standard.

### Example 8

### Preparation of Disinfecting Liquid Formulation

The same protocol adopted in Example 1 was employed to prepare a disinfecting liquid formulation containing the ingredients listed in Table 27 below in the percentage amounts by volume as indicated.

TABLE 27

73% A.R. grade ethanol
10% B.P. grade eucalyptus oil
1.5% “Anhydro Wax”
4% glycerine
11.5% distilled water

### Example 9

### Anti-viral Activity Testing (Herpes Simplex)

The same protocol adopted in Example 2 was employed to determine whether the liquid formulation of Example 8 was veridical against Herpes Simplex Virus Type 1, except that the cell growth medium used was M199 (supplied by Sigma).

### Results

The untreated Herpes control had a log₁₀ titre of 5.7 (see Table 28).

The virus used in the present study was completely inactivated by the test product at the contact times of 5 and 10 minutes at room temperature (see Table 29 and Table 30).

Sample showed no cytotoxicity at the 10⁻² dilution (Table 31).

Product showed signs of complete neutralisation at the 10⁻² dilution (Table 32).

TABLE 28
VIRUS CONTROL RESULTS

| | | |
|---|---|---|
| Virus | | |
| Dilution | No. Inoc. | Individual Responses |

| | | | | | |
|---|---|---|---|---|---|
| 10⁻² | 4 | + | + | + | + |
| 10⁻³ | 4 | + | + | + | + |
| 10⁻⁴ | 4 | + | + | + | + |
| 10⁻⁵ | 4 | + | + | + | + |
| 10⁻⁶ | 4 | + | − | − | − |
| 10⁻⁷ | 4 | − | − | − | − |

| | |
|---|---|
| Total hosts: | 24 |

Calculated virus titre = 10^{5.7} TCID₅₀(5.7 log₁₀)
Note:
Presence of virus in each response is recorded as “+”
Absence virus in each response is recorded as “−”
Cytotoxic response is recorded as “C”

TABLE 29
RESULTS FOR VIRUS TREATED WITH PRODUCT

| Virus | Contact time 5 MINUTES | |
|---|---|---|
| Dilution | No. Inoc. | Individual Responses |

| | | | | | |
|---|---|---|---|---|---|
| 10⁻² | 4 | − | − | − | − |
| 10⁻³ | 4 | − | − | − | − |
| 10⁻⁴ | 4 | − | − | − | − |
| 10⁻⁵ | 4 | − | − | − | − |
| 10⁻⁶ | 4 | − | − | − | − |

| | |
|---|---|
| Total hosts: | 20 |

Calculated virus titre = 10^{1.5} TCID₅₀ (1.5 log₁₀)

TABLE 30
RESULTS FOR VIRUS TREATED WITH PRODUCT

| Virus | Contact time 10 MINUTES | |
|---|---|---|
| Dilution | No. Inoc. | Individual Responses |

| | | | | | |
|---|---|---|---|---|---|
| 10⁻² | 4 | − | − | − | − |
| 10⁻³ | 4 | − | − | − | − |
| 10⁻⁴ | 4 | − | − | − | − |
| 10⁻⁵ | 4 | − | − | − | − |
| 10⁻⁶ | 4 | − | − | − | − |

| | |
|---|---|
| Total hosts: | 20 |

Calculated virus titre = 10^{1.5} TCID₅₀ (1.5 log₁₀)

TABLE 31
RESULTS FOR CYTOTOXICITY CHECK

| | | |
|---|---|---|
| Virus | | |
| Dilution | No. Inoc. | Individual Responses |

| | | | | | |
|---|---|---|---|---|---|
| 10⁻² | 4 | − | − | − | − |
| 10⁻³ | 4 | − | − | − | − |

| | |
|---|---|
| Total hosts: | 8 |

Calculated virus titre = 10^{1.5} TCID₅₀ (1.5 log₁₀)

TABLE 32
RESULTS FOR PRODUCT NEUTRALIZATION

| | | |
|---|---|---|
| Virus | | |
| Dilution | No. Inoc. | Individual Responses |

| | | | | | |
|---|---|---|---|---|---|
| 10⁻² | 4 | + | + | + | + |
| 10⁻³ | 4 | + | + | + | + |
| Total hosts: | 8 | | | | |

Note:
+ represents infected hosts showing haemagglutination
− represents infected hosts showing no haemagglutination
C represents cytotoxic response

TABLE 33
LOG₁₀ REDUCTION OF VIRUS AFTER TREATMENT

| | Titre | Reduction |
|---|---|---|
| Treatment | (Log₁₀) | (Log₁₀) |
| Virus Control | 5.7 | — |
| 5 mins Treatment | 1.5 | 4.2 |
| 10 mins Treatment | 1.5 | 4.2 |

### Conclusions

This study clearly demonstrates that the test product at neat concentration was able to kill Herpes simplex type 1 virus at room temperature with contact times of 5 and 10 minutes in a suspension test model (Table 33). Evidence of complete viral neutralisation after 5 and 10 minute exposure periods, with a 4.2 log reduction in viral titre, indicates that the test at 5 and 10 minute contact times showed compliance with the efficacy requirement for disinfectant as specified in TGO 54 and 54A.

### Example 10

### Anti-Viral Activity Testing (Adeno Virus)

The same protocol adopted in Example 3 was employed to determine whether the liquid formulation of Example 8 was veridical against Adeno type 2 virus.

### Results

The untreated Adeno virus control had a log₁₀ titre of 4.5 (see Table 34).

The virus used in the present study was completely inactivated by the test product at the contact times of 5 and 10 minutes at room temperature (see Table 35 and Table 36).

Sample showed no cytotoxicity at the 10⁻² dilution (Table 37).

Product showed signs of complete neutralisation at the 10⁻² dilution (Table 38).

TABLE 34
VIRUS CONTROL RESULTS

| | | |
|---|---|---|
| Virus | | |
| Dilution | No. Inoc. | Individual Responses |

| | | | | | |
|---|---|---|---|---|---|
| 10⁻² | 4 | + | + | + | + |
| 10⁻³ | 4 | + | + | + | + |
| 10⁻⁴ | 4 | + | + | + | + |
| 10⁻⁵ | 4 | − | − | − | − |
| 10⁻⁶ | 4 | − | − | − | − |
| 10⁻⁷ | 4 | − | − | − | − |
| Total hosts: | 24 | | | | |

Calculated virus titre = 10^{4.5} TCID₅₀(4.5 log₁₀)
Note:
Presence of virus in each response is recorded as “+”
Absence virus in each response is recorded as “−”
Cytotoxic response is recorded as “C”

TABLE 35
RESULTS FOR VIRUS TREATED WITH PRODUCT

| Virus | Contact time 5 MINUTES | |
|---|---|---|
| Dilution | No. Inoc. | Individual Responses |

| | | | | | |
|---|---|---|---|---|---|
| 10⁻² | 4 | − | − | − | − |
| 10⁻³ | 4 | − | − | − | − |
| 10⁻⁴ | 4 | − | − | − | − |
| 10⁻⁵ | 4 | − | − | − | − |
| 10⁻⁶ | 4 | − | − | − | − |
| Total hosts: | 20 | | | | |

Calculated virus titre = 10^{1.5} TCID₅₀ (1.5 log₁₀)

TABLE 36
RESULTS FOR VIRUS TREATED WITH PRODUCT

| Virus | Contact time 10 MINUTES | |
|---|---|---|
| Dilution | No. Inoc. | Individual Responses |

| | | | | | |
|---|---|---|---|---|---|
| 10⁻² | 4 | − | − | − | − |
| 10⁻³ | 4 | − | − | − | − |
| 10⁻⁴ | 4 | − | − | − | − |
| 10⁻⁵ | 4 | − | − | − | − |
| 10⁻⁶ | 4 | − | − | − | − |
| Total hosts: | 20 | | | | |

Calculated virus titre = 10^{1.5} TCID₅₀ (1.5 log₁₀)

TABLE 37
RESULTS FOR CYTOTOXICITY CHECK

| | | |
|---|---|---|
| Virus | | |
| Dilution | No. Inoc. | Individual Responses |

| | | | | | |
|---|---|---|---|---|---|
| 10⁻² | 4 | − | − | − | − |
| 10⁻³ | 4 | − | − | − | − |
| Total hosts: | 8 | | | | |

Calculated virus titre = 10^{1.5} TCID₅₀ (1.5 log₁₀)

TABLE 38
RESULTS FOR PRODUCT NEUTRALIZATION

| | | |
|---|---|---|
| Virus | | |
| Dilution | No. Inoc. | Individual Responses |

| | | | | | |
|---|---|---|---|---|---|
| 10⁻² | 4 | + | + | + | + |
| 10⁻³ | 4 | + | + | + | + |
| Total hosts: | 8 | | | | |

Note:
+ represents infected hosts showing haemagglutination
− represents infected hosts showing no haemagglutination
C represents cytotoxic response

TABLE 39
LOG₁₀ REDUCTION OF VIRUS AFTER TREATMENT

| | Titre | Reduction |
|---|---|---|
| Treatment | (Log₁₀) | (Log₁₀) |
| Virus Control | 4.5 | — |
| 5 mins Treatment | 1.5 | 3.0 |
| 10 mins Treatment | 1.5 | 3.0 |

### Conclusions

This study clearly demonstrates that, the test product at neat concentration, was able to kill Adeno type 2 virus at room temperature with contact times of 5 and 10 minutes in a suspension test model (Table 39). Evidence of viral neutralisation after 5 and 10 minute exposure periods, with a 3.0 log reduction in viral titre, indicates that the test at 5 and 10 minute contact times showed compliance with the efficacy requirement for disinfectant as specified in TGO 54 and 54A.

### Example 11

### Anti-Viral Activity Testing (Human Influenza Virus)

The same protocol adopted in Example 4 was employed to determine whether the liquid formulation of Example 8 was veridical against Human Influenza Virus Type A, except that the contact times used were 5 and 10 minutes.

### Results

The untreated human influenza A (PR8) virus control had a log₁₀ titre of 5.0 (see Table 40).

The virus used in the present study was completely inactivated by the test product at the contact times of 5 and 10 minutes at room temperature (see Table 41 and Table 42).

Sample showed no cytotoxicity at the 10⁻² dilution (Table 43).

Product showed signs of complete neutralisation at the 10⁻² dilution (Table 44).

The washed 0.8% chicken red blood cells settled and formed normal “buttons” in the absence of virus.

TABLE 40
VIRUS CONTROL RESULTS

| | | |
|---|---|---|
| Virus | | |
| Dilution | No. Inoc. | Individual Responses |

| | | | | | |
|---|---|---|---|---|---|
| 10⁻² | 4 | + | + | + | + |
| 10⁻³ | 4 | + | + | + | + |
| 10⁻⁴ | 4 | + | + | + | + |
| 10⁻⁵ | 4 | − | − | + | + |
| 10⁻⁶ | 4 | − | − | − | − |
| 10⁻⁷ | 4 | − | − | − | − |
| Total hosts: | 24 | | | | |

Calculated virus titre = 10^{5.0} TCID₅₀(5.0 log₁₀)
Note:
Presence of virus in each response is recorded as “+”
Absence virus in each response is recorded as “−”
Cytotoxic response is recorded as “C”

TABLE 41
RESULTS FOR VIRUS TREATED WITH PRODUCT

| Virus | Contact time 5 MINUTES | |
|---|---|---|
| Dilution | No. Inoc. | Individual Responses |

| | | | | | |
|---|---|---|---|---|---|
| 10⁻² | 4 | − | − | − | − |
| 10⁻³ | 4 | − | − | − | − |
| 10⁻⁴ | 4 | − | − | − | − |
| 10⁻⁵ | 4 | − | − | − | − |
| 10⁻⁶ | 4 | − | − | − | − |
| Total hosts: | 20 | | | | |

Calculated virus titre = 10^{1.5} TCID₅₀ (1.5 log₁₀)

TABLE 42
RESULTS FOR VIRUS TREATED WITH PRODUCT

| Virus | Contact time 10 MINUTES | |
|---|---|---|
| Dilution | No. Inoc. | Individual Responses |

| | | | | | |
|---|---|---|---|---|---|
| 10⁻² | 4 | − | − | − | − |
| 10⁻³ | 4 | − | − | − | − |
| 10⁻⁴ | 4 | − | − | − | − |
| 10⁻⁵ | 4 | − | − | − | − |
| 10⁻⁶ | 4 | − | − | − | − |
| Total hosts: | 20 | | | | |

Calculated virus titre = 10^{1.5} TCID₅₀ (1.5 log₁₀)

TABLE 43
RESULTS FOR CYTOTOXICITY CHECK

| Virus | | | | | | | |
|---|---|---|---|---|---|---|---|
| Dilution | No. Inoc. | Individual Responses | | | | | |
| 10⁻² | 6 | − | − | − | − | − | − |
| 10⁻³ | 6 | − | − | − | − | − | − |
| Total hosts: | 12 | | | | | | |

Calculated virus titre <10^{1.5}TCID₅₀ (<1.5 log₁₀)

TABLE 44
RESULTS FOR PRODUCT NEUTRALIZATION

| Virus | | | | | | | |
|---|---|---|---|---|---|---|---|
| Dilution | No. Inoc. | Individual Responses | | | | | |
| 10⁻² | 6 | + | + | + | + | + | + |
| 10⁻³ | 6 | + | + | + | + | + | + |
| Total hosts: | 12 | | | | | | |

Note:
+ represents infected hosts showing haemagglutination
− represents infected hosts showing no haemagglutination
C represents cytotoxic response

TABLE 45
LOG₁₀ REDUCTION OF VIRUS AFTER TREATMENT

| | Titre | Reduction |
|---|---|---|
| Treatment | (Log₁₀) | (Log₁₀) |
| Virus Control | 5.0 | — |
| 5 mins Treatment | ≦1.5 | ≧3.5 |
| 10 mins Treatment | ≦1.5 | ≧3.5 |

### Conclusions

This study clearly demonstrates that the test product at neat concentration was able to kill human influenza A completely at room temperature with contact times of 5 and 10 minutes in a suspension test model (Table 45). Evidence of viral neutralisation after 5 and 10 minute exposure periods, with a greater than 3.5 log reduction in viral titre, indicates that the test at 5 and 10 minute contact times showed veridical properties.

### Example 12

### Anti-microbial Activity Challenge Test (TM110)

### Objective

To determine whether the liquid formulation of Example 8 demonstrated anti-microbial activity against Methicillin-resistant Staphylococcus aureus and Vancomycin-resistant Enterococcus faecalis.

### Conditions

Test organisms: 1. Methicillin-resistant Staphylococcus aureus (MRSA)
2. Vancomycin-resistant Enterococcus faecalis (VRE)

### Test Concentration Neat

Contact Times: 30 seconds, 1 minute and 2 minutes

### Test Temperature: Ambient

### Results

TABLE 46
Surviving MRSA after exposure to liquid formulation

| | Surviving organisms (CFU/mL) and Log₁₀ Reduction | | | | | |
|---|---|---|---|---|---|---|
| | 30 sec | | 1 minute | | 2 minutes | |
| | CFU/ | Log | | Log | | Log |
| Sample | mL | Reduc- | CFU/mL | Reduc- | CFU/mL | Reduc- |
| Details | (log₁₀) | tion | (log₁₀) | tion | (log₁₀) | tion |
| Sunny- | <10 | >4.52 | <10 | >4.52 | <10 | >4.52 |
| Wipes | (<1) | | (<1) | | (<1) | |
| Gel | | | | | | |

| | |
|---|---|
| Inoculum | 3.3 × 10⁵ (5.52 Logs) |

CFU = Colony Forming Unit

TABLE 47
Surviving VRE after exposure to liquid formulation

| | Surviving organisms (CFU/mL) and Log₁₀ Reduction | | | | | |
|---|---|---|---|---|---|---|
| | 30 sec | | 1 minute | | 2 minutes | |
| | CFU/ | Log | | Log | | Log |
| Sample | mL | Reduc- | CFU/mL | Reduc- | CFU/mL | Reduc- |
| Details | (log₁₀) | tion | (log₁₀) | tion | (log₁₀) | tion |
| Sunny- | <10 | >4.61 | <10 | >4.61 | <10 | >4.61 |
| Wipes | (<1) | | (<1) | | (<1) | |
| Gel | | | | | | |

| | |
|---|---|
| Inoculum | 4.1 × 10⁵ (5.61 Logs) |

CFU = Colony Forming Unit

### Conclusions

The sample successfully demonstrated anti-microbial activity against Methicillin-resistant Staphylococcus aureus and Vancomycin-resistant Enterococcus faecalis by more than 4 log reduction (kill >99.99%) after 30 seconds, 1 minute and 2 minutes of contact time, when tested neat in conditions described above.

### Example 13

### Anti-microbial Activity Challenge Test (EN 1040:2006)

### Objective

To determine whether the liquid formulation of Example 8 demonstrated bactericidal activity according to the EN 1040:2006 standard.

### Conditions

Test organisms: 1. Staphylococcus aureus ATCC 6538
2. Pseudomonas aeruginosa ATCC 15442

### Inoculum Level; Approx. 1−5×10⁸ Colony Forming Units (CFU)/mL

### Test Concentration Neat

Contact Time: 5 minutes

### Test Temperature Ambient

### Results

TABLE 48
Surviving organisms after 5 minutes exposure to liquid formulation

| | Surviving organisms (CFU/mL) and Log₁₀ Reduction | | | |
|---|---|---|---|---|
| | S. aureus | | Ps. aeruginosa | |
| | CFU/mL | Log | CFU/mL | Log |
| Sample | (log₁₀) | Reduction | (log₁₀) | Reduction |
| Antimicrobial | <140 | >5.48 | <140 | >5.23 |
| Gel | (<2.15) | | (<2.15) | |

| | | |
|---|---|---|
| In test Inoculum | 4.3 × 10⁷ | 2.4 × 10⁷ |
| | (7.63) | (7.38) |

Notes:
1. Product Neutralisation—The validated neutraliser was T6 (a mixture of Tryptone Soy Broth, Lecithin and Tween 80) for both organisms tested.
2. All controls and validation were satisfactory.

### Conclusions

The sample successfully demonstrated bactericidal activity according to the EN 1040:2006 standard. The sample showed more than 5.48 log reduction against Staphylococcus aureus ATCC 6538 and more than 5.23 log reduction against Pseudomonas aeruginosa ATCC 15442 when tested neat in conditions described above. The sample requires 5 minutes of contact time to meet the accepted criteria for making bactericidal claims according to those required by the EN 1040:2006 standard.

### Example 14

### Evaluation of Trans-epidermal Water Loss

### Objective

The effect of the liquid formulation of Example 8 on skin hydration was evaluated using a TEWA Meter and compared with untreated skin on the same test panelists at 30 min, 2 days, 4 days and 7 days.

### Standards for Inclusion in a Study

1. Individuals between the ages of 18 and 70.
2. Individuals not taking medication or under the care of a physician for a period of one month prior to commencement and throughout the entire test period.
3. Individuals who have completed a preliminary medical history mandated by Dermatest.
4. Individuals who have read, understood and signed an informed consent document. Consent forms are kept on file and are available for examination on the premises of Dermatest.
5. Individuals who understand the instructions for use and are willing to cooperate with the program as stated.
6. Individuals free of any dermatological or systemic disorder that would interfere with the results, at the discretion of the Investigator.
7. Individuals able to cooperate with the Investigator and the research staff and willing to complete the full course of the study.
   Standards for Exclusion from a Study
1. Individuals who are under doctors' care.
2. Individuals who are currently taking medication which in the opinion of the Investigator would mask or interfere with the results.
3. Individuals with any history of sensitivity to cosmetics in general and moisturizers in particular.
4. Individuals with any form of skin cancer, melanoma, lupus, psoriasis, rosacea, porphyria cutanea tarda, connective tissue disease, or any disease that would interfere with the test results.
5. Individuals diagnosed with chronic skin allergies.
6. Female volunteers who indicate that they are pregnant or nursing an infant.
7. Individuals with excessive hair on the test sites.
8. Individuals with known hypersensitivity to cosmetic products.

### Informed Consent

A signed informed consent was obtained from each panelist prior to initiating the study describing reasons for the study, possible adverse effects, associated risks and potential benefits of the treatment and their limits of liability. Each subject was assigned a permanent identification number and completed an extensive medical history form. These forms along with the signed consent forms are available for inspection on the premises of Dermatest.

### Institutional Ethics Committee

The IEC of Dermatest Pty Ltd consists of 5 or more individuals, chosen in accordance with ICH Guidelines for Good Clinical Practice. The list of IEC members is kept on file at Dermatest Pty Ltd and is available for inspection on the premises during normal office hours.

### Methodology

One distinct method was employed for the evaluation procedure. Biophysical measurements were made pre-application (t=0) and following a single application of the test material after 30 min. Additional readings were also taken at 2 days, 4 days and 7 days. Before each set of measurements, subjects were required to equilibrate in a closed environment with constant temperature (20° C.+/−2° C.).

### Moisture Measurement—Corneometer

Model TM 210 TEWA Meter—Courage+Khazala

### REFERENCES

Tewameter T M 210 Information and Operating Instructions (manual),
Transepidermal Water Loss, Bioengineering of the Skin: Methods and Instrumentation, CRC Press 1995.
Dermatest SOP DESOP—030 Procedure for Determining Transepidermal Water Loss (TEWL).

### Study Design

5 healthy panelists between the ages of 42 to 60 years were inducted into this study. In order to precondition the test sites and keep all topical treatments constant for all test subjects, panelists were required to abstain from using deodorant soaps, moisturizing soaps or cosmetic moisturizers on the test area for a period of one week prior to study commencement and during the course of the study. At the completion of the one week ‘washout’ period, panelists were required to return to the test facility at the time specified by the technician for the study commencement. On the day of the study, test material was delivered to the test sites by applying to the back of the hands; hands were washed liberally and then rinsed off under water. This was repeated 10 times a day for 7 days, at 1 hour intervals during daylight hours. Panelists were blinded as to the nature of the material being applied, Biophysical measurements with a TEWA Meter were taken at t=0 (pre-application). Panelists were required to return to the lab at each subsequent designated period for repeat biophysical measurements.

### Results

TEWL change from t=0 (pre-application) to t=30 min (first wash): 5%

TEWL change from t=0 (pre-application) to t=7 days (final wash): 7%

### Conclusions

After rigorous repeat applications over a period of 7 days, the reduction in trans-epidermal water loss was minimal and there was no incidence of skin irritation under the test conditions. There was no significant difference between the initial loss at first wash and the loss at 7 days. There was no indication that regular use reduces skin integrity. No adverse effects or unexplained reactions of any kind were observed on any of the subjects.

### Example 15

### Skin Irritation Testing

### 1.0 Objective Consumer products or raw materials designed for consistent reapplication to areas of the skin may, under proper conditions, prove to be contact sensitizers or irritants in certain individuals. It is the intention of a Repeat Insult Patch Test (RIPT) to provide a basis for evaluation of this irritation/sensitization potential if such exists.

### 2.0 Reference

The method is modified to test 50 panelists and not the 200 cited in the reference Appraisal of the Safety of Chemicals in Food, Drugs and Cosmetics, published by the Association of Food and Drug Officials of The United States. The method also employs nine inductive patchings and not the ten cited in the reference under semi-occlusive patch conditions.

### 3.0 Test Material

### 3.1 Test Material Description

Wipes (assigned CR Lab No. E0730-A) comprising ethanol and eucalyptus oil were tested.

### 4.0 Panel Selection

### 4.1 Standards for Inclusion in a Study

Individuals who are not currently under a doctor's care.
Individuals free of any dermatological or systemic disorder which would interfere with the results, at the discretion of the Investigator.
Individuals free of any acute or chronic disease that might interfere with or increase the risk of study participation.
Individuals who will complete a preliminary medical history form and are in general good health.
Individuals who will read, understand and sign an informed consent document relating to the specific type of study they are subscribing.
Individuals able to cooperate with the Investigator and research staff, willing to have test materials applied according to the protocol, and complete the full course of the study.

### 4.2 Standards for Exclusion from a Study

Individuals under 18 years of age.
Individuals who are under doctor's care.
Individuals who are currently taking any medication (topical or systemic) that may mask or interfere with the test results.
Subjects with a history of any acute or chronic disease that might interfere with or increase the risk of study participation.
Individuals diagnosed with chronic skin allergies.
Female volunteers who indicate that they are pregnant or nursing.

### 4.3 Recruitment

Panel selection is accomplished by advertisement in local periodicals, community bulletin boards, phone solicitation, electronic media or any combination thereof.

### 4.4 Informed Consent and Medical History Forms

An informed consent was obtained from each volunteer prior to initiating the study describing reasons for the study, possible adverse effects, associated risks and potential benefits of the treatment and their limits of liability. Panelists signed and dated the informed consent document to indicate their authorization to proceed and acknowledge their understanding of the contents. Each subject was assigned a permanent identification number and completed an extensive medical history form.

### 5.0 Population Demographics

Number of subjects enrolled . . . 52
Number of subjects completing study . . . 52

### Age Range . . . 19-65

### Sex . . . Male . . . 13

Female . . . 39

### Race . . . Caucasian . . . 30

Hispanic . . . 10
Asian . . . 2
African American . . . 10

### 6.0 Equipment

Patch Description: Parke-Davis Hypoallergenic Readi Bandages (20×20 mm Webril affixed to the centre of a 40×40 mm adhesive bandage) or the equivalent, trimmed at right angles on opposite sides to the opening of the paper backing of patch, allowing air flow.
1 ml volumetric syringe without a needle.

### 7.0 Procedure

Subjects are requested to bathe or wash as usual before arrival at the facility.
0.2 ml of the test material was dispensed onto a semi-occlusive, hypoallergenic patch.
The patch was then affixed directly to the skin of the infrascapular regions of the back, to the right or left of the midline and the subject was dismissed with instructions not to wet or expose the test area to direct sunlight.
After 24 hours the patch was removed by the panelist at home.
This procedure was repeated until a series of nine consecutive 24 hour exposures have been made for every Monday, Wednesday and Friday for three consecutive weeks.
In the event of an adverse reaction, the area of erythema and edema is measured. The edema is estimated by the evaluation of the skin with respect to the contour of the unaffected normal skin. Reactions are scored just before applications two through nine and the next test date following application nine. Clients are notified immediately in the case of adverse reaction and determination is made as to treatment program if necessary.
Subjects were then given a 10-14 day rest period after which a challenge or retest dose was applied once to a previously unexposed test site. The retest dose is equivalent to any one of the original nine exposures. Reactions are scored 24 and 48 hours after application.
Comparison was made between the nine sensitizing doses and the retest dose.
At the end of the study, the consulting Dermatologist revised this data and confirmed the stated conclusions.

### 8.0 Results

| | Subject | | | Response | | | | | | | | | Chall. | |
|---|---|---|---|---|---|---|---|---|---|---|---|---|---|---|
| No. | ID | RACE | SEX | 1 | 2 | 3 | 4 | 5 | 6 | 7 | 8 | 9 | 24 HR | 48 HR |
| 1 | 03-7001 | C | M | 0 | 0 | 0 | 0 | 0 | 0 | 0 | 0 | 0 | 0 | 0 |
| 2 | 03-7079 | C | F | 0 | 0 | 0 | 0 | 0 | 0 | 0 | 0 | 0 | 0 | 0 |
| 3 | 03-7269 | C | F | 0 | 0 | 0 | 0 | 0 | 0 | 0 | 0 | 0 | 0 | 0 |
| 4 | 03-6521 | C | F | 0 | 0 | 0 | 0 | 0 | 0 | 0 | 0 | 0 | 0 | 0 |
| 5 | 03-7077 | C | F | 0 | 0 | 0 | 0 | 0 | 0 | 0 | 0 | 0 | 0 | 0 |
| 6 | 03-7297 | C | F | 0 | 0 | 0 | 0 | 0 | 0 | 0 | 0 | 0 | 0 | 0 |
| 7 | 03-6679 | C | M | 0 | 0 | 0 | 0 | 0 | 0 | 0 | 0 | 0 | 0 | 0 |
| 8 | 03-6919 | AA | F | 0 | 0 | 0 | 0 | 0 | 0 | 0 | 0 | 0 | 0 | 0 |
| 9 | 03-7241 | C | F | 0 | 0 | 0 | 0 | 0 | 0 | 0 | 0 | 0 | 0 | 0 |
| 10 | 03-7245 | C | F | 0 | 0 | 0 | 0 | 0 | 0 | 0 | 0 | 0 | 0 | 0 |
| 11 | 03-6643 | C | F | 0 | 0 | 0 | 0 | 0 | 0 | 0 | 0 | 0 | 0 | 0 |
| 12 | 03-7261 | H | F | 0 | 0 | 0 | 0 | 0 | 0 | 0 | 0 | 0 | 0 | 0 |
| 13 | 03-7294 | H | F | 0 | 0 | 0 | 0 | 0 | 0 | 0 | 0 | 0 | 0 | 0 |
| 14 | 03-7295 | H | F | 0 | 0 | 0 | 0 | 0 | 0 | 0 | 0 | 0 | 0 | 0 |
| 15 | 03-6906 | C | F | 0 | 0 | 0 | 0 | 0 | 0 | 0 | 0 | 0 | 0 | 0 |
| 16 | 03-7262 | C | F | 0 | 0 | 0 | 0 | 0 | 0 | 0 | 0 | 0 | 0 | 0 |
| 17 | 03-6040 | C | F | 0 | 0 | 0 | 0 | 0 | 0 | 0 | 0 | 0 | 0 | 0 |
| 18 | 03-6883 | H | M | 0 | 0 | 0 | 0 | 0 | 0 | 0 | 0 | 0 | 0 | 0 |
| 19 | 03-6019 | A | F | 0 | 0 | 0 | 0 | 0 | 0 | 0 | 0 | 0 | 0 | 0 |
| 20 | 03-7128 | C | M | 0 | 0 | 0 | 0 | 0 | 0 | 0 | 0 | 0 | 0 | 0 |
| 21 | 03-7270 | C | F | 0 | 0 | 0 | 0 | 0 | 0 | 0 | 0 | 0 | 0 | 0 |
| 22 | 03-7247 | H | F | 0 | 0 | 0 | 0 | 0 | 0 | 0 | 0 | 0 | 0 | 0 |
| 23 | 03-7256 | C | F | 0 | 0 | 0 | 0 | 0 | 0 | 0 | 0 | 0 | 0 | 0 |
| 24 | 03-6391 | AA | F | 0 | 0 | 0 | 0 | 0 | 0 | 0 | 0 | 0 | 0 | 0 |
| 25 | 03-6003 | C | F | 0 | 0 | 0 | 0 | 0 | 0 | 0 | 0 | 0 | 0 | 0 |
| 26 | 03-6065 | C | M | 0 | 0 | 0 | 0 | 0 | 0 | 0 | 0 | 0 | 0 | 0 |
| 27 | 03-7189 | C | F | 0 | 0 | 0 | 0 | 0 | 0 | 0 | 0 | 0 | 0 | 0 |
| 28 | 03-7279 | AA | M | 0 | 0 | 0 | 0 | 0 | 0 | 0 | 0 | 0 | 0 | 0 |
| 29 | 03-7291 | C | F | 0 | 0 | 0 | 0 | 0 | 0 | 0 | 0 | 0 | 0 | 0 |
| 30 | 03-6416 | AA | F | 0 | 0 | 0 | 0 | 0 | 0 | 0 | 0 | 0 | 0 | 0 |
| 31 | 03-7024 | AA | F | 0 | 0 | 0 | 0 | 0 | 0 | 0 | 0 | 0 | 0 | 0 |
| 32 | 03-7085 | C | F | 0 | 0 | 0 | 0 | 0 | 0 | 0 | 0 | 0 | 0 | 0 |
| 33 | 03-6045 | A | M | 0 | 0 | 0 | 0 | 0 | 0 | 0 | 0 | 0 | 0 | 0 |
| 34 | 03-6720 | AA | F | 0 | 0 | 0 | 0 | 0 | 0 | 0 | 0 | 0 | 0 | 0 |
| 35 | 03-6770 | C | F | 0 | 0 | 0 | 0 | 0 | 0 | 0 | 0 | 0 | 0 | 0 |
| 36 | 03-7237 | C | F | 0 | 0 | 0 | 0 | 0 | 0 | 0 | 0 | 0 | 0 | 0 |
| 37 | 03-6078 | C | M | 0 | 0 | 0 | 0 | 0 | 0 | 0 | 0 | 0 | 0 | 0 |
| 38 | 03-6933 | AA | M | 0 | 0 | 0 | 0 | 0 | 0 | 0 | 0 | 0 | 0 | 0 |
| 39 | 03-7260 | C | M | 0 | 0 | 0 | 0 | 0 | 0 | 0 | 0 | 0 | 0 | 0 |
| 40 | 03-7288 | H | F | 0 | 0 | 0 | 0 | 0 | 0 | 0 | 0 | 0 | 0 | 0 |
| 41 | 03-6039 | C | F | 0 | 0 | 0 | 0 | 0 | 0 | 0 | 0 | 0 | 0 | 0 |
| 42 | 03-6176 | AA | F | 0 | 0 | 0 | 0 | 0 | 0 | 0 | 0 | 0 | 0 | 0 |
| 43 | 03-7080 | C | F | 0 | 0 | 0 | 0 | 0 | 0 | 0 | 0 | 0 | 0 | 0 |
| 44 | 03-6998 | AA | F | 0 | 0 | 0 | 0 | 0 | 0 | 0 | 0 | 0 | 0 | 0 |
| 45 | 03-6518 | C | F | 0 | 0 | 0 | 0 | 0 | 0 | 0 | 0 | 0 | 0 | 0 |
| 46 | 03-6398 | AA | M | 0 | 0 | 0 | 0 | 0 | 0 | 0 | 0 | 0 | 0 | 0 |
| 47 | 03-6634 | C | F | 0 | 0 | 0 | 0 | 0 | 0 | 0 | 0 | 0 | 0 | 0 |
| 48 | 03-6174 | C | F | 0 | 0 | 0 | 0 | 0 | 0 | 0 | 0 | 0 | 0 | 0 |
| 49 | 03-6356 | H | M | 0 | 0 | 0 | 0 | 0 | 0 | 0 | 0 | 0 | 0 | 0 |
| 50 | 03-6164 | H | M | 0 | 0 | 0 | 0 | 0 | 0 | 0 | 0 | 0 | 0 | 0 |
| 51 | 03-6583 | H | F | 0 | 0 | 0 | 0 | 0 | 0 | 0 | 0 | 0 | 0 | 0 |
| 52 | 03-6071 | H | F | 0 | 0 | 0 | 0 | 0 | 0 | 0 | 0 | 0 | 0 | 0 |

### 9. Observations

No adverse reactions of any kind were noted during the course of this study.

### 10. Conclusions

The test material when tested under semi-occlusive conditions as described herein, may be considered as a non-primary irritant and a non-primary sensitizer to the skin according to the reference.

### Example 16

### Comparative Example

### Sample Description

Hard surface carrier tests were conducted to compare the anti-bacterial activities of a water added test solution (A) and a test solution (B) without added water.

“Solution A”, 73:18:9 (Ethanol:Eucalyptus Oil (BP Grade):Water v/v/v)

Sample tested as received.

### Examination

Hard Surface Carrier Test Dilution: Neat

### Method

AOAC Method 991.47, 991.48 and 991.49 (Salmonella choleraesuis, Staphylococcus aureus and Pseudomonas aeuruginosa)

### Conditions

### Temperature: 20±2.0° C.

### Concentration: Neat

### Soil: Organic (5% Horse Serum)

### Contact Time: 10 min

### Results

| | | | CARRIERS | TOTAL | |
|---|---|---|---|---|---|
| | ATCC REF | SUB- | WITH | INOCULATED | ACCEPTANCE |
| ORGANISM | # | CULTURE | GROWTH | CARRIERS | CRITERIA* |
| Salmonella | 10708 | 6 | 0 | 60 | 2/60 |
| choleraesuis | | | | | |
| Staphylococcus | 6538 | 6 | 1 | 60 | 2/60 |
| aureus | | | | | |
| Pseudomonas | 15442 | 6 | 0 | 60 | 3/60 |
| aeruginosa | | | | | |

*Maximum allowable number of carriers showing growth out of total number inoculated.

### Control Results

### Carrier Organism Recovery

| | Duplicate Counts from Individual | | | | | | Mean |
|---|---|---|---|---|---|---|---|
| | Carriers (cfu at 10⁻⁵ dilution) | | | | | | cfu/ |
| | 1 | 2 | 3 | 4 | 5 | 6 | carrier |
| Salmonella | 40/41 | 25/15 | 29/31 | 45/35 | 18/18 | 61/52 | 3.4 × 10⁶ |
| choleraesuis | | | | | | | |
| Staphylococcus | 54/79 | 18/18 | 24/17 | 19/17 | 29/20 | 18/18 | 2.7 × 10⁶ |
| aureus | | | | | | | |
| Pseudomonas | 27/23 | 27/33 | 20/15 | 39/28 | 18/18 | 22/22 | 2.4 × 10⁶ |
| aeruginosa | | | | | | | |

cfu = colony forming units

These carrier recoveries conform to the method requirements.

### Product Neutralisation

The validated neutralizer was T6 (a mixture of Tryptone Soy Broth, Lecithin and Tween 80). This was confirmed prior to actual testing.

### Sample Description

“Solution B”, 80:20 (Ethanol:Eucalyptus Oil (BP Grade))

Sample tested as received.

### Examination

Hard Surface Carrier Test Dilution: Neat

### Method

AOAC Method 991.47, 991.48 and 991.49 (Salmonella choleraesuis, Staphylococcus aureus and Pseudomonas aeuruginosa)

### Conditions

### Temperature: 20±2.0° C.

### Concentration: Neat

### Soil: Organic (5% Horse Serum)

### Contact Time: 10 min

### Results

| | | | CARRIERS | TOTAL | |
|---|---|---|---|---|---|
| | ATCC REF | SUB- | WITH | INOCULATED | ACCEPTANCE |
| ORGANISM | # | CULTURE | GROWTH | CARRIERS | CRITERIA* |
| Salmonella | 10708 | 8 | 3 | 60 | 2/60 |
| choleraesuis | | | | | |
| Staphylococcus | 6538 | 8 | 15 | 60 | 2/60 |
| aureus | | | | | |
| Pseudomonas | 15442 | 8 | 13 | 60 | 3/60 |
| aeruginosa | | | | | |

*Maximum allowable number of carriers showing growth out of total number inoculated.

### Control Results

### Carrier Organism Recovery

| | Duplicate Counts from Individual | | | | | | Mean |
|---|---|---|---|---|---|---|---|
| | Carriers (cfu at 10⁻⁵ dilution) | | | | | | cfu/ |
| | 1 | 2 | 3 | 4 | 5 | 6 | carrier |
| Salmonella | 36/33* | 37/33* | 73/69* | 62/54* | 65/70* | 75/105* | 5.9 × 10⁵ |
| choleraesuis | | | | | | | |
| Staphylococcus | 55/58 | 65/52 | 39/52 | 61/42 | 36/48 | 40/61 | 5.1 × 10⁶ |
| aureus | | | | | | | |
| Pseudomonas | 22/22 | 28/23 | 16/20 | 18/17 | 23/24 | 146/145* | 2.0 × 10⁶ |
| aeruginosa | | | | | | | |

cfu = colony forming units

These carrier recoveries conform to the method requirements.

### Product Neutralisation:

The validated neutralizer was T6 (a mixture of Tryptone Soy Broth, Lecithin and Tween 80). This was confirmed prior to actual testing.

### CONCLUSION

“Solution A”, when tested according to the conditions described herein, met the requirement of AOAC Hard Surface Carrier Test for all three test organisms, namely, Salmonella choleraesuis, Staphylococcus aureus and Pseudomonas aeruginosa. However, “Solution B”, without added water, did not meet the AOAC Hard Surface Carrier Test requirement for any of these test organisms, indicating that inclusion of water has a significant effect on anti-bacterial activity.

It will be appreciated that the present invention has been described by way of example only and that modifications and additions may be made thereto without departing from the scope of the invention as defined in the appended claims.

1. A disinfecting formulation comprising:
(a) alcohol;
(b) one or more essential oils comprising cineole;
(c) gelling agent; and
(d) water.

2. The formulation according to claim 1 wherein said alcohol comprises one or more C₁ to C₁₀ alcohol.

3. The formulation according to claim 2 wherein said alcohol comprises one or more of methanol, ethanol and isopropanol.

4. The formulation according to claim 3 wherein said alcohol comprises ethanol of analytical grade (A.R).

5. The formulation according to any one of the preceding claims wherein said one or more essential oils is selected from eucalyptus, tea tree, bayleaf, spearmint and rosemary oils.

6. The formulation according to claim 5 wherein said one or more essential oils comprises eucalyptus oil.

7. The formulation according to claim 6 wherein said eucalyptus oil is of B.P. (British, Pharmacopoeia) grade.

8. The formulation according to claim 1, further comprising clove oil and/or sweet orange oil.

9. The formulation according to claim 1, wherein said gelling agent is selected from one or more of vegetable, animal, mineral, petroleum or synthetic waxes, vegetable gums, starches, pectins, gelatine, chitin, chitosan, collagen, silica, cornstarch, glycols and carbomer (polyacrylic acid).

10. The formulation according to claim 9 wherein said vegetable gums are selected from locust bean gum, guar gum, xanthan gum, alginates, agar, carageenan, betaglucan, gellan gum, gum arabic, gum tragacanth, karaya gum, locust bean gum, mastic gum, psyllium gum, spruce gum, ghatti gum and glucomannan.

11. The formulation according to claim 9 wherein said vegetable, animal, mineral, petroleum or synthetic waxes are selected from beeswax, shellac wax, spermaceti, lanolin, bayberry wax, candelilla wax, carnauba wax, castor wax, esparto wax, jojoba oil, ouricury wax, rice bran wax, soy wax, ceresin waxes, montan wax, ozocerite, paraffin wax, microcrystalline wax, polyethylene waxes, chemically modified waxes, Fischer-Tropsch waxes, substituted amide waxes and polymerised a-olefins.

12. The formulation according to claim 9 wherein said gelling agent is a petroleum wax.

13. The formulation according to claim 12 wherein said gelling agent is “Anhydro Wax”.

14. The formulation according to claim 1, wherein the ratio, by volume, of water to essential oil is up to about 1.5:1.

15. The formulation according to claim 1, comprising, by volume, from about 0.1% to about 5% gelling agent, from about 30% to about 85% alcohol and from about 5% to about 30% essential oil.

16. The formulation according to claim 15 comprising, by volume, from about 0.5% to about 3% gelling agent, from about 60% to about 80% alcohol and from about 10% to about 25% essential oil.

17. The formulation according to claim 16 comprising, by volume, from about 1% to about 2% gelling agent, from about 70% to about 75% alcohol and from about 10% to about 15% essential oil.

18. The formulation according to claim 1, further comprising one or more emollient agents.

19. The formulation according to claim 18 wherein said one or more emollient agents is selected from lanolin, mineral, vegetable and synthetic oils and humectants.

20. The formulation according to claim 19 wherein said vegetable oils are selected from coconut oil, jojoba oil, shea butter, mango butter and palm oil.

21. The formulation according to claim 19 wherein said humectants are selected from glycerine, propylene glycol, glyceryl triacetate, sorbitol, xylitol, melitol and polydextrose.

22. The formulation according to any one of claims 18 to 21 comprising, by volume, from about 1 to about 2% gelling agent, about 72% alcohol, about 11% essential oil, from about 1 to about 2% emollient agent and about 14% water.

23. A disinfecting formulation comprising, by volume:
(a) from about 65% to about 75% ethanol;
(b) from about 10% to about 15% eucalyptus oil;
(c) from about 0.5% to about 2% “Anhydro Wax”;
(d) from about 1% to about 4% glycerine; and
(e) water.

24. The formulation according to claim 23 wherein the ratio, by volume, of water to eucalyptus oil is up to about 1.5:1.

25. The formulation according to either claim 23 or claim 24 comprising, by volume:
(a) about 72% ethanol;
(b) about 11% eucalyptus oil;
(c) from about 1 to about 2% “Anhydro Wax”;
(d) from about 1 to about 2% glycerine; and
(e) about 14% water.

26. The formulation according to either claim 23 or claim 24 comprising, by volume:
(a) about 73% ethanol;
(b) about 10% eucalyptus oil;
(c) about 1.5% “Anhydro Wax”;
(d) about 4% glycerine; and
(e) about 11.5% water.

27. A method of disinfecting a human or animal body part comprising applying to the body part a formulation according to any one of the preceding claims.

28. A method of disinfecting a human or animal body part comprising applying to the body part a formulation comprising, by volume:
(a) from about 65% to about 75% ethanol;
(b) from about 10% to about 15% eucalyptus oil;
(c) from about 0.5% to about 2% “Anhydro Wax”;
(d) from about 1% to about 4% glycerine; and
(e) water.

29. The method according to claim 28 wherein the formulation comprises, by volume:
(a) about 72% ethanol;
(b) about 11% eucalyptus oil;
(c) from about 1 to about 2% “Anhydro Wax”;
(d) from about 1 to about 2% glycerine; and
(e) about 14% water.

30. The method according to claim 28 wherein the formulation comprises, by volume:
(a) about 73% ethanol;
(b) about 10% eucalyptus oil;
(c) about 1.5% “Anhydro Wax”;
(d) about 4% glycerine; and
(e) about 11.5% water.

31. The method according to any one of claims 27 to 30 for disinfecting human hands.

32. A method of preparing a disinfecting formulation according to any one of claims 23 to 26 which comprises combining said “Anhydro Wax” and eucalyptus oil to form a dispersion, which is then added to a mixture of said ethanol, glycerine and water with gentle mixing to produce the disinfecting formulation.
